(19) United States
(12) Patent Application Publication
(10) Pub. No.: US 2026/0263536 A1
(43) Pub. Date: Sep. 10, 2026

(54) USE OF LACTIPLANTIBACILLUS PLANTARUM GOLDGUT-HNU082 IN ALLEVIATING SIDE EFFECTS OF ANTITUMOR DRUG

(71) Applicant: SHENZHEN PORSHEALTH BIOENGINEERING CO., LTD., SHENZHEN (CN)

(72) Inventors: GUOXUN XIAO, SHENZHEN (CN); SILU ZHANG, SHENZHEN (CN); SHAN LU, SHENZHEN (CN); XIAOLI ZHOU, SHENZHEN (CN); SHUYING ZHONG, SHENZHEN (CN); LE LIU, SHENZHEN (CN)

(21) Appl. No.: 19/343,096

(22) Filed: Sep. 29, 2025

(30) Foreign Application Priority Data
Mar. 6, 2025 (CN) 202510259055.2

(51) Int. Cl. A61K 35/744; A61K 35/00; A61P 35/00

(52) U.S. Cl. CPC A61K 35/744; A61P 35/00; A61K 2035/115

(57) ABSTRACT

Uses of Lactiplantibacillus plantarum GOLDGUT-HNU082 in alleviating the side effects of an antitumor drug, preparing a bacterial agent for alleviating side effects of gefitinib, a targeted drug for non-small cell lung cancer, or preparing a medicament for inhibiting non-small cell lung cancer cells, and a microbial agent containing Lactiplantibacillus plantarum GOLDGUT-HNU082 are provided. The deposit number of the Lactiplantibacillus plantarum GOLDGUT-HNU082 is CGMCC NO. 33075.

### CROSS REFERENCE TO RELATED APPLICATIONS

The present disclosure claims the priority to the Chinese patent application with the filing No. 2025102590552, entitled “USE OF LACTIPLANTIBACILLUS PLANTARUM GOLDGUT-HNU082 IN ALLEVIATING SIDE EFFECTS OF ANTITUMOR DRUG” and filed on Mar. 6, 2025 with the Chinese Patent Office, the contents of which are incorporated herein by reference in their entirety.

### TECHNICAL FIELD

The present disclosure relates to the field of microbial technology, and more particularly to a use of Lactiplantibacillus plantarum GOLDGUT-HNU082 in alleviating side effects of an antitumor drug.

### BACKGROUND ART

The gut is the largest immune organ in the human body, governing 70% or more of the human body's immune functions and generating approximately 80% of the resistance. Probiotics, as beneficial bacteria in the gut, have various functions such as regulating the gut microbiota, promoting nutrient absorption, boosting body's immunity, suppressing intestinal inflammation, and protecting the intestinal mucosal barrier. Despite their numerous benefits, probiotics may also interact with drugs, leading to some adverse effects. Probiotics may influence their host's individualized response to drug therapy through means such as drug degradation. In some cases, the intake of probiotics may accelerate the chemical transformation of drugs, thereby affecting the drugs'effects at specific positions. For example, studies have shown that probiotics may degrade the lipid-lowering drug lovastatin, but this effect does not necessarily affect the efficacy of the drug; instead, it may have a beneficial effect of alleviating liver inflammation. However, this does not mean that all interactions between probiotics and all drugs are harmless.

In recent years, the incidence of lung cancer has shown an upward trend. Gefitinib, one of the targeted drugs for treating lung cancer, has good safety and tolerability; and as an oral epidermal growth factor receptor tyrosine kinase inhibitor, it inhibits the activity of epidermal growth factor receptor tyrosine kinase, thereby hindering tumor growth, metastasis and angiogenesis, and increasing tumor cell apoptosis. Gefitinib has a high oral bioavailability up to 60% to 80%. It is rapidly absorbed in the gastrointestinal tract, primarily metabolized by the liver and excreted in the bile, with only a small portion excreted in the urine in an unmetabolized form. However, gefitinib may also cause some adverse reactions in the digestive system, such as diarrhea and elevated transaminases. However, the presence of probiotics may have a positive impact on the therapeutic efficacy of gefitinib by alleviating its side effects, protecting gut health, and enhancing immune function, but this has not yet been publicly reported.

Therefore, exploring probiotics for alleviating the side effects of gefitinib is an urgent problem that needs to be solved by those skilled in the art.

### SUMMARY

In view of this, the present disclosure provides a use of Lactiplantibacillus plantarum GOLDGUT-HNU082 in alleviating the side effects of gefitinib, a targeted drug for non-small cell lung cancer.

In order to achieve the above purpose, the present disclosure adopts the following technical solutions.

The use of Lactiplantibacillus plantarum GOLDGUT-HNU082 in alleviating the side effects of gefitinib, a targeted drug for non-small cell lung cancer, where the Lactiplantibacillus plantarum GOLDGUT-HNU082 has a deposit number of CGMCC NO. 33075.

Another purpose of the present disclosure is to provide a use of Lactiplantibacillus plantarum GOLDGUT-HNU082 in the preparation of a bacterial agent for alleviating the side effects of gefitinib, a targeted drug for non-small cell lung cancer, where the deposit number of the Lactiplantibacillus plantarum GOLDGUT-HNU 082 is CGMCC NO. 33075.

Another purpose of the present disclosure is to provide a microbial agent, where the microbial agent includes Lactiplantibacillus plantarum GOLDGUT-HNU082 with a deposit number of CGMCC NO. 33075.

Preferably, the microbial agent has the effect of alleviating the side effects of gefitinib, a targeted drug for non-small cell lung cancer.

Another purpose of the present disclosure is to provide a use of Lactiplantibacillus plantarum GOLDGUT-HNU082 in the preparation of a drug for inhibiting non-small cell lung cancer cells, where the deposit number of the Lactiplantibacillus plantarum GOLDGUT-HNU082 is CGMCC NO. 33075.

Beneficial Effects: gefitinib is a targeted therapy drug for EGFR gene-mutated non-small cell lung cancer with clear efficacy, but its side effects limit its application. The Lactiplantibacillus plantarum GOLDGUT-HNU 082 (deposit number of CGMCC NO. 33075) of the present disclosure can significantly alleviate liver and gut damage caused by gefitinib, as evidenced by decreased liver transaminases, reduced inflammatory markers, and more intact liver and intestinal tissue structure. Furthermore, the bacterium also significantly increases the production of short-chain fatty acids, further confirming its regulatory effect on the gut microbiome. This study aims to explore the mechanism by which probiotics mediate the gut microbiota to alleviate drug side effects, promote probiotics to become a companion to gefitinib, reduce patients' psychological burden of taking the drug, and significantly alleviate drug side effects.

### BRIEF DESCRIPTION OF DRAWINGS

In order to more clearly illustrate the technical solutions of the embodiments of the present disclosure or in the prior art, the drawings required for use in description of the embodiments or the prior art will be briefly introduced below. Obviously, the drawings described below are merely embodiments of the present disclosure. For a person ordinarily skilled in the art, other drawings may be obtained based on the provided drawings without paying any creative work.

FIG. 1 is a colony morphology diagram of Lactiplantibacillus plantarum GOLDGUT-HNU082.

FIG. 2 shows the whole genome comparison results of Lactiplantibacillus plantarum GOLDGUT-HNU082.

FIG. 3 shows the degradation rate of gefitinib by different lactic acid bacteria.

FIG. 4 shows the effect of Lactiplantibacillus plantarum GOLDGUT-HNU082 metabolites on non-small cell lung cancer cells HCC827.

FIG. 5 shows the effect of Lactiplantibacillus plantarum GOLDGUT-HNU082 on the tumor volume in mice with lung cancer.

FIG. 6 shows the effect of Lactiplantibacillus plantarum GOLDGUT-HNU082 on tumor weight in mice with lung cancer.

FIG. 7 shows the effects of Lactiplantibacillus plantarum GOLDGUT-HNU082 on liver function indices in mice with lung cancer.

FIG. 8 shows the effects of Lactiplantibacillus plantarum GOLDGUT-HNU082 on the liver and intestinal tissues in mice with lung cancer.

FIG. 9 shows the effects of Lactiplantibacillus plantarum GOLDGUT-HNU082 on the contents of short-chain fatty acids in the intestine in mice with lung cancer.

### DETAILED DESCRIPTION OF EMBODIMENTS

The technical solutions in the embodiments of the present disclosure will be clearly and completely described below in conjunction with the drawings in the embodiments of the present disclosure. Obviously, the described embodiments are only some of the embodiments of the present disclosure, not all of the embodiments. Based on the embodiments of the present disclosure, all other embodiments obtained by a person ordinarily skilled in the art without making creative efforts are within the scope of protection of the present disclosure.

Examples of the present disclosure discloses the use of Lactiplantibacillus plantarum GOLDGUT-HNU082 in alleviating the side effects of gefitinib, a targeted drug for non-small cell lung cancer. The experimental drug gefitinib (secondary pharmaceutical standard) involved in the present disclosure was purchased from AstraZeneca UK Limited; the HCC827 cells involved were human non-small cell lung cancer cells, purchased from the cell resource platform established by Xiamen Immocell Biotechnology Co., Ltd.; the Lactobacillus paracasei H22B008, Lactobacillus paracasei H22B022 , Lactiplantibacillus plantarum E22D048, Ligilactobacillus salivarius F22C005, and Ligilactobacillus salivarius F22E058 involved were all purchased from the Hainan University Tropical Probiotic Lactic Acid Bacteria Collection Center. Unless otherwise specified, the methods involved in the present disclosure were all conventional methods and may not be described in detail herein.

All statistical analyses in the present disclosure were performed by using Graphpad Pism 9.5 software for one-way ANOVA. Significant differences were considered at a p-value threshold of 0.05 (*p<0.05, **p<0.01). Data were presented as mean±standard error (SE).

Graphs were plotted by using Graphpad Pism 9.5.

### EXAMPLE 1

### Isolation, Screening and Identification of Lactiplantibacillus Plantarum GOLDGUT-HNU082

1. 30 fermented food samples were collected for subsequent use from farmers' markets in various counties and cities in Hainan Province, including Wanning, Wenchang, Wuzhishan, Baisha, and Lingshui, etc. These collected fermented food samples were diluted tenfold in sterilized physiological saline and subsequently prepared into sample dilutions at gradients of: 10⁻¹, 10⁻², 10⁻³, 10⁻⁴, and 10⁻⁵ by using a ten-fold dilution method. 0.1 mL of 10⁻³, 10⁻⁴, and 10⁻⁵ sample dilutions (sample dilutions at the gradients of 10⁻³, 10⁻⁴, and 10⁻⁵) were respectively spread onto a MRS agar medium, cultured anaerobically at 37° C. for 48 h, and subjected to multiple times of isolation, streaking, purification, culturing, and identification, to screen out 5 Lactiplantibacillus plantarum strains. The 5 strains were cultured in a MRS liquid for 24 h, and the OD value and viable bacteria count were measured, and finally, a Lactiplantibacillus plantarum strain was screened out and named GOLDGUT-HNU082. The purified GOLDGUT-HNU082 strain was inoculated into a MRS broth medium and activated for 24 h, and then spread onto a MRS agar medium, and after culturing at 37° C. for 24 h, the bacterial colony was white, had a diameter of 0.1-0.2 cm, and had a smooth, moist, raised surface and a neat edge (as shown in FIG. 1). When observed under an optical microscope, the bacteria were rod-shaped.

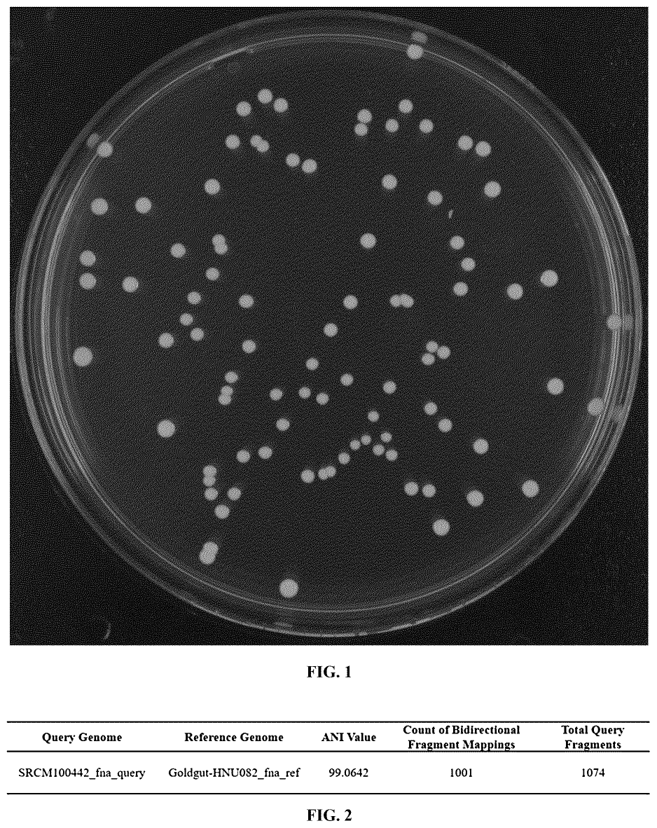

2. Identification Information: the GOLDGUT-HNU 082 strain was subjected to whole genome sequencing. Sequences obtained from whole genome sequencing were subjected to genomic alignment. Genomic similarity analysis was performed by using the online analysis platform Galaxy (https://usegalaxy.org/) and FastANI software (https://github.com/ParBLiSS/FastANI). The analysis results showed that the ANI value of the whole genome sequence of GOLDGUT-HNU082 and that of Lactiplantibacillus plantarum SRCM100442 was 99.0642 (as shown in FIG. 2).

3. The strain was deposited in the China General Microbiological Culture Collection Center (CGMCC), with the deposit address at the Institute of Microbiology, Chinese Academy of Sciences, No. 3, Yard 1, Beichen West Road, Chaoyang District, Beijing. The deposit number was CGMCC NO. 33075, the deposit date was Dec. 16, 2024, and the taxonomic name was Lactiplantibacillus plantarum.

### EXAMPLE 2

### Effects of Lactiplantibacillus plantarum GOLDGUT-HNU082 on Gefitinib

### 1. Degradation Rate of Gefitinib

Based on previous studies, the concentration of gefitinib stock solution was determined to be 33 μM. The medium, bacterial solution, and gefitinib stock solution were mixed uniformly at a ratio of 12 mL: 400 μL:200 μL, co-cultured for 0 h, 6 h, 12 h, and 24 h, and the expression peak area was determined by using a high-performance liquid chromatograph from Agilent Technologies Co., Ltd. (under the following conditions: chromatographic column: octadecylsilane-bonded silica gel column; column temperature: 30° C.; mobile phase: ammonium acetate solution: acetonitrile=65:35; flow rate: 1.0 mL/min; detection: 243 nm ultraviolet detection). Taking the gefitinib cultured alone in the medium as a blank control, the degradation rate of gefitinib by probiotics was calculated by comparing the peak area of gefitinib co-cultured with probiotics with that of gefitinib alone.

The different types of probiotics were Lactiplantibacillus plantarum GOLDGUT-HNU082, Lactobacillus paracasei H22B008, Lactobacillus paracasei H22B022, Lactiplantibacillus plantarum E22D048, Ligilactobacillus salivarius F22C005, and Ligilactobacillus salivarius F22E058.

The calculation formula for the degradation rate of gefitinib was as follows:

V=(A−B)/A

V: degradation rate of gefitinib by probiotics

A: peak area of gefitinib

B: peak area of gefitinib after co-culturing with probiotics

The results were as shown in FIG. 3. Among different probiotics, Lactiplantibacillus plantarum GOLDGUT-HNU082 had the lowest degradation rate of gefitinib.

### 2. Effects on Non-Small Cell Lung Cancer Cells

HCC827 cells were grown in Dulbecco's Modified Eagle Medium (DMEM), to which 10% fetal bovine serum was added, and to which antibiotics (1% penicillin and 1% streptomycin) were supplemented. Cells were cultured in a fully humidified, 5% CO₂ incubator at 37° C. All experiments were performed on cells in the logarithmic growth phase. The medium was replaced every 2 days, and cells were subcultured by using 0.25% phenol red-free trypsin.

After trypsin digestion and cell collection, 1 ml of medium was added and mixed to prepare a suspension. Subsequently, cell counting was performed, and they were adjusted to approximately 2000 cells per milliliter.

After cell counting, a 96-well culture plate was prepared. HCC827 cells were then evenly distributed into each well of the plate and the culture plate was placed in a 37° C. incubator. After 48 hours of incubation, during which the cells grew normally and reached confluence, the probiotics were inoculated onto MRS broth medium. When the probiotics culture reached the maximum growth phase, the supernatant was collected by centrifugation at a rotation speed of 6000 rpm for a time of 3 min. Subsequently, the supernatant was taken out on a clean bench, sterilized by using a 0.22 μM microporous filter membrane, and stored at −80° C. The Lactiplantibacillus plantarum GOLDGUT-HNU082 metabolite supernatant was diluted with cell medium as the diluting agent to sequentially precisely prepare, according to different concentrations, Lactiplantibacillus plantarum GOLDGUT-HNU082 metabolite solutions with concentrations of 10 μM, 15 μM, 20 μM, 25 μM, and 30 μM. After preparation, each concentration of metabolite solution was precisely added to the corresponding cell culture wells respectively according to the experimental design, ensuring accuracy and consistency in the addition amount to meet the requirements of different concentration conditions for subsequent cell experiments. The culture plates were then incubated for an additional 24 h, and cell viability was assessed by using the MTT assay. After 24 h of cell treatment, the culture medium was carefully removed, and 100 μL of MTT dye solution was added to each well. The plate was then placed in the incubator for an additional 4 to 6 h. After incubation, 150 μL of DMSO was added to each well and shaken for 10 min to fully dissolve the crystals. The absorbance (OD) at a wavelength of 570 nm was then measured by using a microplate reader (Tecan Infinite® 200 PRO, Switzerland).

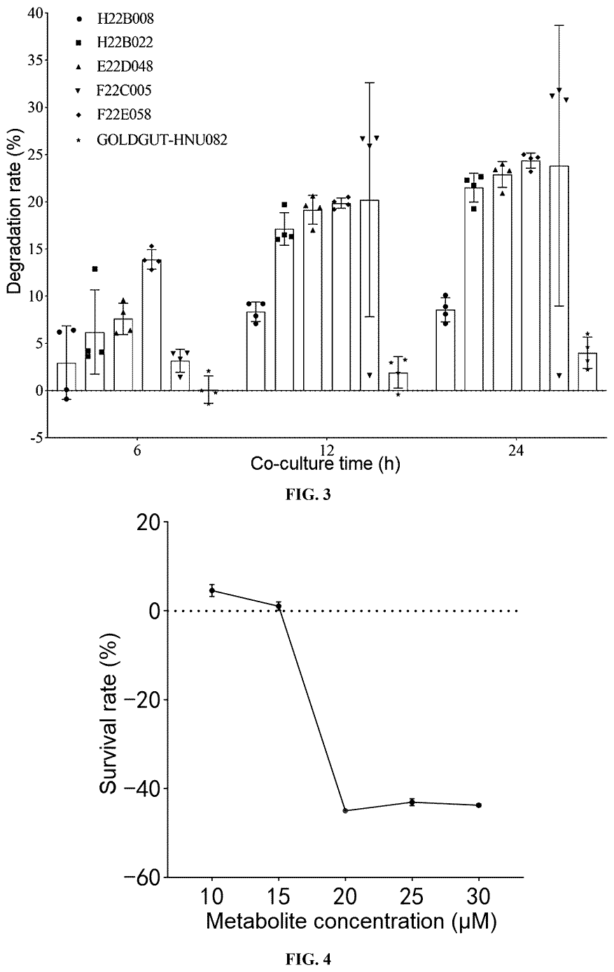

The results were as shown in FIG. 4. When coexisting with 20 μM of Lactiplantibacillus plantarum GOLDGUT-HNU082 metabolites, the survival rate of lung cancer cells quickly reached the lowest point. This result suggests that Lactiplantibacillus plantarum GOLDGUT-HNU082 had a significant inhibitory effect on HCC827 cells.

### 3. Effects on Lung Cancer Mice

### (1) Methods:

This study was approved by the Animal Ethics Committee of Hainan University, and all animal procedures were performed in accordance with the Guidelines for the Care and Use of Laboratory Animals of Hainan University. Twenty-four female SPF-grade BALB/c Nude nude mice (4 weeks old) were purchased from Beijing Vital River Laboratory Animal Technology Co., Ltd. The nude mice were raised under standard laboratory conditions with a 12/12 h light/dark cycle, 60% humidity, and a temperature of 23° C., with unrestricted access to a standard food diet and drinking water. The nude mice were allowed to acclimate to the environment for one week before the experiment. Both the control group and lung cancer model group were fed a basic maintenance diet provided by Beijing Keao Xieli Feed Co., Ltd.

HCC827 cells were adjusted by using 1×PBS and Matrigel at a ratio of 1:1 to obtain a suspension with a concentration of 1×10⁷ cells per milliliter. The cell suspension was placed on ice and then rapidly injected subcutaneously near the left hind leg of nude mice, with each mouse receiving an injection volume of 200 μL. Fourteen days after HCC827 cell inoculation, obvious masses were palpable at the inoculation site, indicating that the tumor cells had successfully proliferated and formed tumors in the nude mice, and the modeling was successful. Mice with successful model establishment were administrated with gefitinib for treatment, with appropriate dosage and course of administration being set. Indices such as changes in tumor volume and body weight of the mice were observed and recorded. If, after gefitinib treatment, the tumor growth in the mice was significantly inhibited, with the tumor volume shrinking or the growth rate slowing down remarkably, and the body weight of the mice remained relatively stable or there were no obvious adverse reactions, it would indicate that the modeled mice had a good therapeutic response to gefitinib, which is in line with the indications of gefitinib. Body weight and tumor volume were recorded regularly thereafter. Tumor volume was calculated as (L×W×H)/2, where L was the tumor length, W was the tumor width, and H was the height.

When the nude mice's tumor volume exceeded 50 mm³, they were grouped according to the tumor volume. This study included four groups (n=6): a blank control group (Control,CON); a non-small cell lung cancer model group (Model,MO); a gefitinib-treated non-small cell lung cancer model group (Model+Gefitinib, Gef); and a non-small cell lung cancer model group treated with combination of gefitinib and Lactiplantibacillus plantarum GOLDGUT-HNU082 (Model+Gefitinib+Lactiplantibacillus plantarum GOLDGUT-HNU082, G+HNU082).

After grouping, a 6-week treatment for the nude mice was started. The CON and MO groups were given 200 μL of 0.5% sodium carboxymethyl cellulose solution by gavage daily; the Gef group was given 200 μL of 100 mg/kg gefitinib (dissolved using 0.5% sodium carboxymethyl cellulose) by gavage daily; and the G+HNU082 group was given 200 μL of 100 mg/kg gefitinib +10⁹ cfu Lactiplantibacillus plantarum GOLDGUT-HNU082 (dissolved using 0.5% sodium carboxymethyl cellulose) by gavage daily. Meanwhile, their body weight was measured once a week, and their tumor volume was measured once a week.

After 6 weeks of treatment, the effect of the presence of probiotics on the therapeutic effect of gefitinib was determined.

Feces were collected from the mice once before the treatment and once after the treatment ended, and immediately stored at −80° C. for metagenomic determination. After 6 weeks of treatment, blood, tissue samples, and colonic contents were collected from the mice under sterile conditions. Serum was obtained by centrifugation of blood. The serum and colonic contents were stored at −80° C. until use for the determination of transaminases and inflammatory factors of the nude mice. Serum was used to measure transaminases, bilirubin, creatinine, and other indices, while colonic contents were used to measure short-chain fatty acids. The dissected liver was divided into two parts: one part was placed in a sterile centrifuge tube for quick freezing in liquid nitrogen and then stored at −80°; and the other part was rinsed with 0.85% physiological saline and fixed in paraformaldehyde for liver tissue sectioning (Wuhan Servicebio Technology Co., Ltd.). The dissected small intestine was fixed in paraformaldehyde for intestinal sectioning (Wuhan Servicebio Technology Co., Ltd.). Short-chain fatty acids were analyzed and determined by using gas chromatography-mass spectrometry (GC-MS) from Agilent Technologies Co., Ltd. (column: Agilent DB-WAX: 0.25 mm×0.25 μm×50 cm; inlet temperature: 250° C.; gas interface temperature: 250° C.; carrier gas flow rate: 1.5 mL/min; split ratio=3:1; split ratio: 1 μL).

### (2) Results:

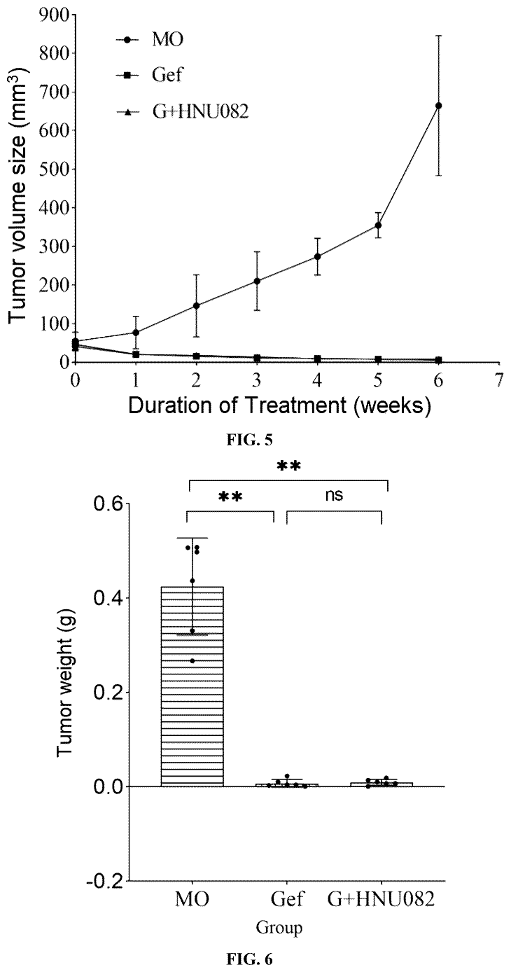

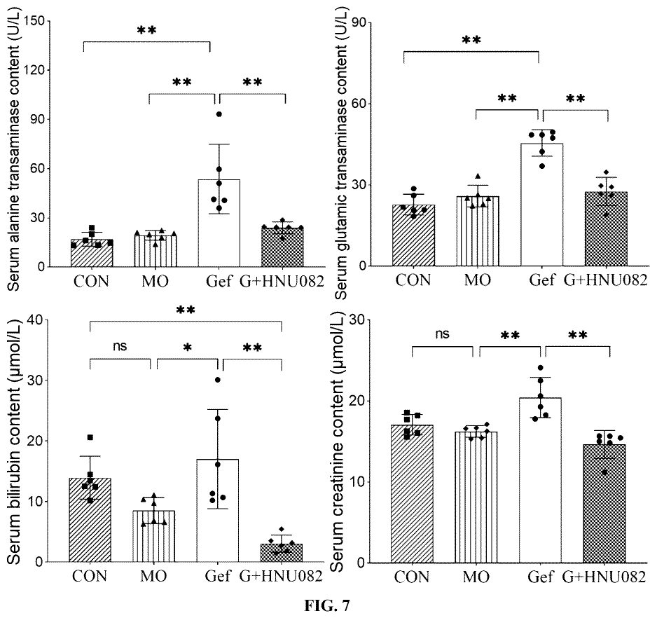

Tumor volume and weight: the results were as shown in FIG. 5 and FIG. 6. After 6 weeks of treatment, there was no significant difference in tumor volume and tumor weight between the gefitinib group (Gef) and a gefitinib-Lactiplantibacillus plantarum GOLDGUT-HNU082 combination group (G+HNU082), indicating that the simultaneous intake of Lactiplantibacillus plantarum GOLDGUT-HNU082 did not affect the therapeutic effect of the drug gefitinib in the treatment of lung cancer.

Liver function indices: the results were as shown in FIG. 7. Compared with the model group, levels of serum indices in the gefitinib group, including alanine transaminase, glutamic transaminase, and serum creatinine were significantly elevated (p<0.01), and total bilirubin was also significantly elevated (p<0.05). However, compared with the gefitinib group, the gefitinib-Lactiplantibacillus plantarum GOLDGUT-HNU082 combination group significantly reduced levels of alanine transaminase, glutamic transaminase, total bilirubin, and serum creatinine in serum (p<0.01). This suggested that Lactiplantibacillus plantarum GOLDGUT-HNU082 significantly mitigated gefitinib-induced liver injury.

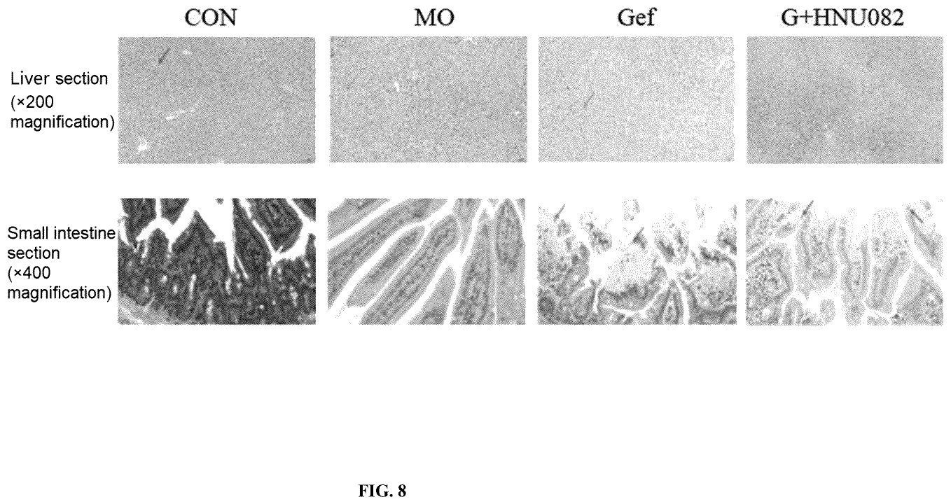

Liver and intestinal tissue sections: as shown in FIG. 8, liver tissue section results showed that the hepatocyte ballooning in the gefitinib-Lactiplantibacillus plantarum GOLDGUT-HNU082 combination group was less than that in the gefitinib group; and in intestinal tissue section results, the gefitinib-Lactiplantibacillus plantarum GOLDGUT-HNU082 combination group showed significantly less lymphocyte infiltration than the gefitinib group. This indicated that gefitinib treatment of non-small cell lung cancer may induce inflammatory responses in the liver and intestine of nude mice. However, when gefitinib was administered in combination with the probiotic Lactiplantibacillus plantarum GOLDGUT-HNU082, these inflammatory responses were effectively alleviated.

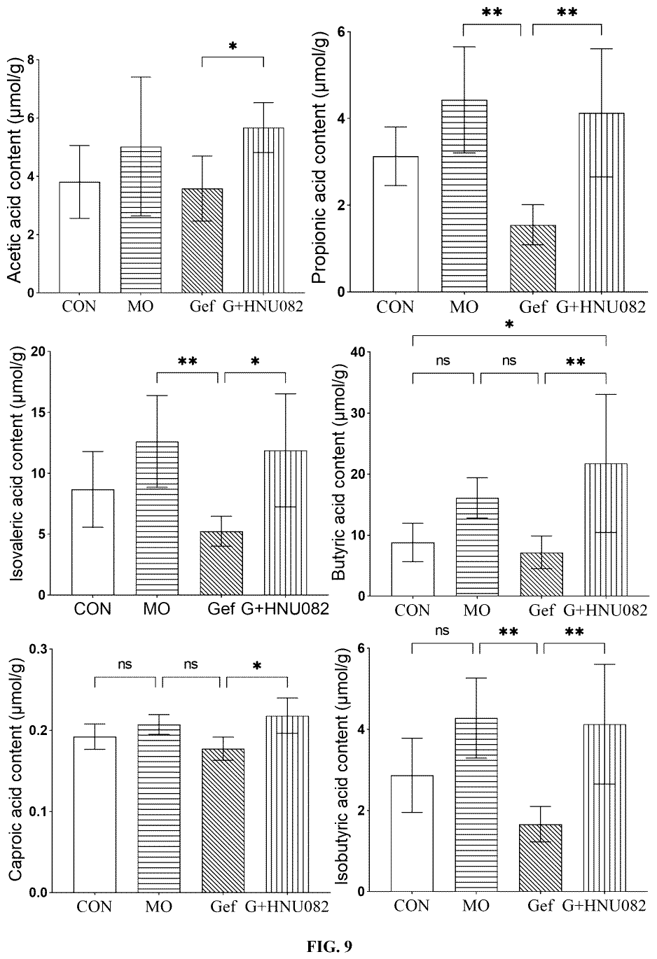

Intestinal short-chain fatty acid content: as shown in FIG. 9, the gefitinib-Lactiplantibacillus plantarum GOLDGUT-HNU082 combination group showed significantly higher contents of acetic acid, isovaleric acid, and caproic acid than the gefitinib group (p<0.05); and the gefitinib-Lactiplantibacillus plantarum GOLDGUT-HNU082 combination group also showed extremely significantly higher contents of propionic acid, butyric acid, and isobutyric acid than the gefitinib group (p<0.01). This suggested that supplementing of Lactiplantibacillus plantarum GOLDGUT-HNU082 during gefitinib treatment of lung cancer may, to a certain extent, increase the content of short-chain fatty acids in the body. Short-chain fatty acids, as products of gut microbial fermentation of indigestible carbohydrates, played a key role in maintaining gut health. Physiologically, it precisely regulated the proliferation and differentiation of intestinal epithelial cells, maintaining the intestinal barrier and defending against pathogens. Immunologically, it influenced immune cell activity and cytokine secretion, enhancing immunity and reducing inflammation. For example, butyric acid inhibited the expression of inflammatory factors. Meanwhile, short-chain fatty acids regulated gut microbial communities, created a suitable environment for beneficial bacteria, inhibited harmful bacteria, and reduced harmful metabolites. For example, propionic acid protected gut health and participated in the regulation of the gut nervous system to maintain normal digestive function.

The various embodiments in this specification are described in a progressive manner, and each embodiment focuses on the differences from other embodiments. The same or similar parts between the various embodiments may be referenced to each other.

The above description of the disclosed embodiments is intended to enable one skilled in the art to implement or use the present disclosure. Various modifications to these embodiments will be apparent to one skilled in the art, and the general principles defined herein may be implemented in other embodiments without departing from the spirit or scope of the present disclosure. Therefore, the present disclosure is not limited to the embodiments shown herein but is intended to conform to the widest scope consistent with the principles and novel features disclosed herein.

1. A method for alleviating side effects of gefitinib, a targeted drug for non-small cell lung cancer, comprising administering Lactiplantibacillus plantarum GOLDGUT-HNU082 to a subject, wherein a deposit number of the Lactiplantibacillus plantarum GOLDGUT-HNU082 is CGMCC NO. 33075.

2. A method for preparing a bacterial agent for alleviating side effects of gefitinib, a targeted drug for non-small cell lung cancer, or a medicament for inhibiting non-small cell lung cancer cells, comprising using Lactiplantibacillus plantarum GOLDGUT-HNU082 to prepare the bacterial agent for alleviating side effects of gefitinib or the medicament for inhibiting non-small cell lung cancer cells, wherein a deposit number of the Lactiplantibacillus plantarum GOLDGUT-HNU082 is CGMCC NO. 33075.

3. A microbial agent, comprising Lactiplantibacillus plantarum GOLDGUT-HNU082 with a deposit number of CGMCC NO. 33075.

4. The microbial agent according to claim 3, wherein the microbial agent has an effect of alleviating side effects of gefitinib, a targeted drug for non-small cell lung cancer.
